(19) United States
(12) Patent Application Publication
(10) Pub. No.: US 2023/0024192 A1
(43) Pub. Date: Jan. 26, 2023

(54) Citrate-Functionalized Polymeric Surfactants Based Upon Alkyl Polyglucosides

(71) Applicant: Colonial Chemical, Inc., South Pittsburg, TN (US)

(72) Inventors: Robert J. Coots, South Pittsburg, TN (US); Dennis Abbeduto, Jr., South Pittsburg, TN (US); Matt Grochowski, South Pittsburg, TN (US); Robert N. Comber, South Pittsburg, TN (US)

(21) Appl. No.: 17/777,958

(22) PCT Filed: Nov. 25, 2020
(86) PCT No.: PCT/US20/62184

§ 371 Date: May 18, 2022

Related U.S. Application Data
(60) Provisional application No. 62/940,093, filed on Nov. 25, 2019.

(51) Int. Cl. C07H 15/04; A61K 8/60; A61Q 5/02

(52) U.S. Cl. CPC C07H 15/04; A61K 8/602; A61Q 5/02

(57) ABSTRACT

The invention relates to a citrate-functionalized polyglucoside derivatives, and crosslinked variants thereof, that are made by the reaction of an epichlorohydrin/citric acid ester with an alkyl polyglycoside. These functionalized alkyl polyglucosides are viscosity builders and anti-irritants in personal care cleansing products.

### PRIOR APPLICATIONS

This application claims benefit to U.S. Patent Application No. 62/940,093, filed Nov. 25, 2019, the contents of which are incorporated herein by reference.

### FIELD OF THE INVENTION

One aspect of the present invention relates to a functionalized polyglycoside derivative that is made by the polymerized reaction of a polyglycoside, together with a functionalizing agent that contains a citrate ester group. The preferred polymers are cross-linked, having more than one group per molecule. The citrate-functionalized alkylpolyglucosides of the present invention are derived entirely from renewable plant resources and are low-irritation, anionic surfactants with excellent foam and viscosity building properties.

In one embodiment, the present invention relates to a polyglycoside derivative that are (a) polymeric (that is cross linked with hydroxypropyl linkages) and (b) contains an additional functional group of a citrate ester. These polymers have higher molecular weight than the starting materials. Among other benefits, the inclusion of the additional citrate ester functional groups provides increased foam.

Commercial alkyl polyglycosides generally have a low degree of polymerization of polysaccharide, in the molecule. This results in a molecule that is of limited water solubility. The present invention is aimed at functionalizing the relatively hydrophobic alkyl polyglycoside, by including in the polymeric molecule specific functionalities. These starting glucosides have been called “alkyl glycosides, alkyl glycosides, alkyl polyglycosides or alkyl polyglycosides” by many different authors. All refer to the same molecules.

### BACKGROUND OF THE INVENTION

Alkyl polyglycosides (APGs) have been known for many years, having been first synthesized in the early 1900 by Emile Fischer. Despite this, the products were of little commercial interest until much later.

U.S. Pat. No. 4,393,203 issued Jul. 12, 1983 to Mao et al, discloses that long chain fatty alcohols used in their syntheses can be removed from alkyl polysaccharide products in thin film evaporators to achieve fatty alcohol levels of less than about 2% without excessive discoloration of the alkyl polysaccharide. This allowed for a more cosmetically acceptable product to be developed that is more surface active. The low level of the free fatty alcohol in the mixture allows for a more water-soluble product, by removing the water insoluble alcohol.

U.S. Pat. No. 5,003,057 issued Mar. 26, 1991, to McCurry et al., provides for a process for preparing glycosides from a source of saccharide moiety and an alcohol in the presence of a hydrophobic acid catalyst is provided. An example of such a catalyst is dinonylnaphthalenemonosulfonic acid. The use of such catalysts provides a number of process advantages, which includes the reduced production of polar by-products. Preferred glycosides produced by the process are higher alkyl glycosides useful as surfactants.

U.S. Pat. No. 3,598,865 (Lew) discloses the production of higher alkyl (C₈-C₂₅) glycosides from a monosaccharide or source thereof and a higher monohydric alcohol in the presence of a latent solvent (lower alcohols) and an acid catalyst selected from the group consisting of sulfuric acid, hydrochloric acid, phosphoric acid, phosphorous acid, toluenesulfonic acid, and boron trifluoride.

U.S. Pat. No. 3,219,656 (Boettner) discloses a process for producing a higher alkyl glycoside by reacting glucose with methanol in the presence of a macroreticular-structured sulfonic acid resin, anhydrous and in the acid form, to produce methyl glycoside which is reacted without isolation with butanol to form butyl glycoside and which in turn is reacted with a higher alcohol to form a surface active higher alkyl glycoside.

U.S. Pat. No. 3,839,319 (Mansfield) discloses a process for producing alkyl glycosides by direct, acid catalyzed reaction of a higher alcohol and a saccharide. The acid catalysts are mineral acids such as hydrochloric and sulfuric, and sulfonic acid exchange resins.

The compounds known before the current invention have been primarily used in industrial applications like detergents for dish wash. This is due in part to inherent drying that occurs when these materials are applied to the skin. Many people, one of which is Cognis, have introduced blends of alkyl polyglycosides and traditional surfactants to overcome these limitations. The blending of other alternative surfactants, while showing improvement in the performance of the product, does not address underlying difficulties in the molecule.

None of the patents referenced above provide for a molecule that has the necessary crosslinking or functionalization incorporated into the molecule as to overcome the negatives associated with the use of the raw alkyl glycoside.

One of the first invention to do so is described in U.S. Pat. No. 7,507,399 (O'Lenick, Jr.), which discloses a series of multifunctional polyglycoside derivatives that are made by the polymerization of 1,3-dichloro-2-propanol and polyglycosides, together with functionalizing agents that contain a sulfonate, quaternary nitrogen or a phosphate group. A number of other derivatized APG's are described in the following patents: sulfonate derivatized alkyl polyglucosides (U.S. Pat. No. 6,627,612, surfactants sold by Colonial Chemical, Inc. under the brand names Suga®Nate), phosphate derivatized alkyl polyglucosides (U.S. Pat. No. 6,627,612, surfactants sold by Colonial Chemical, Inc. under the brand names Suga®Fax) amphoteric glycinate derivatized alkyl polyglucosides (U.S. Pat. No. 6,958,315 surfactants sold by Colonial Chemical under the brand name Suga®Glycinate), and sulfosuccinate derivatized alkyl polyglucosides (U.S. Pat. No. 7,87,571, surfactants sold by Colonial Chemical, Inc. under the brand name Suga®Mates),

Other examples of derivatized alkyl polyglucosides include polysulfonate derivatized alkyl polyglucosides (U.S. Pat. No. 7,507,399, surfactants sold by Colonial Chemical, Inc. under the brand names Poly Suga®Nates), polyphosphate derivatized alkyl polyglucoside (U.S. Pat. No. 7,507,399, surfactants sold by Colonial Chemical, Inc. under the brand names Poly Suga®Phos) polyquaternary derivatized alkyl polyglucosides (U.S. Pat. No. 7,507,399, surfactants sold by Colonial Chemical, Inc. under the brand names Poly Suga®Quats), polycarboxylated derivatized alkyl polyglucosides (U.S. Pat. No. 7,335,627, surfactants sold by Colonial Chemical, Inc. under the brand name Poly Suga®Carb), and polynonionic derivatized alkyl polyglucosides (U.S. Pat. No. 8,268,766, surfactants sold by Colonial Chemical, Inc. under the brand names PolySuga®Mulse).

### BRIEF DESCRIPTION OF THE FIGURES

FIG. 1 shows the viscosity building effects of disodium laurylglucosides hydroxypropyl citrate in SLS

FIG. 2 shows the viscosity building effects of disodium laurylglucosides hydroxypropyl citrate in sodium C₁₄₋₁₆ olefin sulfonate (AOS).

FIG. 3 shows irritation potential of an Anionic Cleanser as predicted by the Zein Test.

### SUMMARY OF THE INVENTION

The present inventors have surprisingly learned that taking the alkyl polyglycosides produced in the commercial process, with its inherent poor water solubility and reacting it to make cross linked polymers, and include functional groups, including citrate ester groups, results in a series of products that are much more usable in many applications.

Thus, one aspect of the present invention relates to the finding that the reaction of the rather hydrophobic alkyl polyglycosides with a functionalizing agent formed between citric acid and epichlorohydrin provides surfactants useful as primary or secondary surfactants useful in shampoo, body washes, facial washes, or other cosmetics.

Another aspect of the present invention is a citrate-functionalized polyglucoside derivatives, and crosslinked variants thereof, that are made by the reaction of an epichlorohydrin/citric acid ester with an alkyl polyglycoside. The functionalized alkyl polyglucosides of the present invention can act as viscosity builders and anti-irritants in personal care cleansing products.

Another aspect of the present invention is a citrate-functionalized alkyl polyglucoside composition.

Another aspect of the present invention is a personal care product, such as a shampoo, body wash, facial wash, conditioning rinse, or other cosmetic composition that comprises the citrate-functionalized alkyl polyglucoside composition.

One embodiment of the present invention is a composition comprising a component of the following formula:

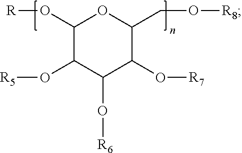

wherein R is a C₁-C₂₄ alkyl; n is an integer from 1 to 4; R₅, R₆, R₇, and R₈ are independently chosen from H or the following:

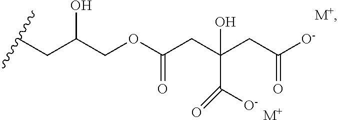

with the proviso that R₅, R₆, R₇, and R₈ are not all H; and M is selected from Na, K, and NH₄; and positional isomers thereof.

Another embodiment of the present invention is a composition comprising a component of the following formula:

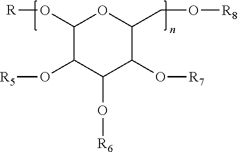

wherein R is a C₁-C₂₄ alkyl; n is an integer from 1 to 4; R₅, R₆, R₇, and R₈ are independently selected from H or the following:

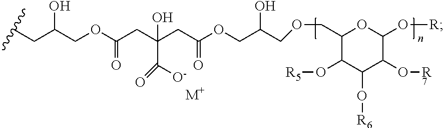

with the proviso that R₅, R₆, R₇, and R₈ are not all H; and M is selected from Na, K, and NH₄; and positional isomers thereof.

Another embodiment of the present invention is a surfactant composition of the following formula:

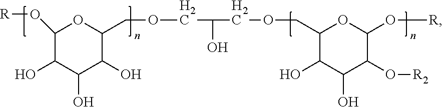

wherein R is C₁-C₂₄ alkyl and R² is

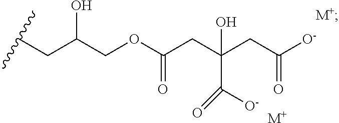

and n is an integer from 1 to 4; M is selected from Na, K, and NH₄; and positional isomers thereof.

Another embodiment of the present invention is a surfactant composition of the following formula:

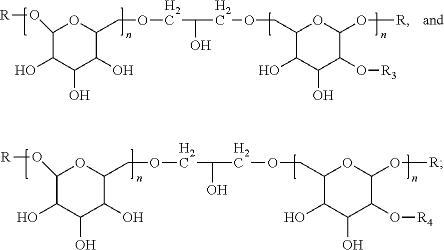

wherein R is C₁-C₂₄ alkyl; and R₃ and R₄ are attachment points for the following functionalizing linker group:

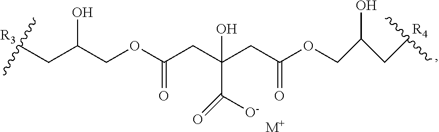

and M is selected from Na, K, and NH₄; n is an integer from 1 to 4; and positional isomers thereof.

One embodiment of the present invention is a composition prepared by reacting the following:

(i) at least one compound of the following formula:

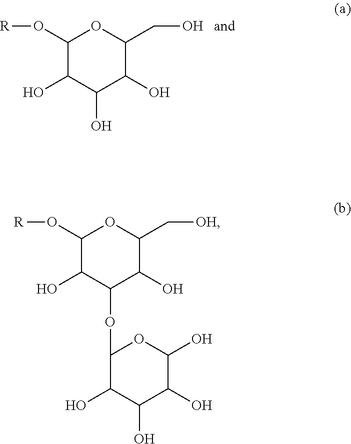

wherein R is an alkyl having 1 to 24 carbons; and

(ii) at least one functionalizing agent of the following formula

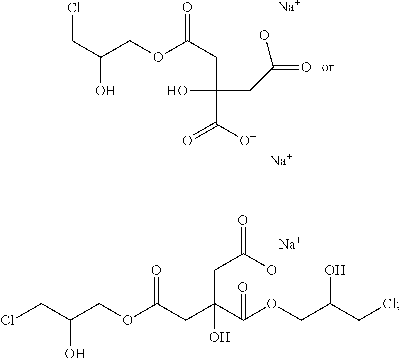

in water, with NaOH, at a temperature of between 75-85° C., for between 5-8 hours.

In another embodiment of the present invention, the composition is prepared by further reacting with at least one polymerizing agent of the following formula:

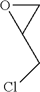

or Cl—CH₂CH(OH)—CH₂— Cl.

Another embodiment of the present invention is a surfactant composition of the following formula:

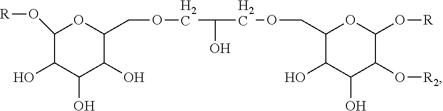

wherein R is C₁-C₂₄ alkyl and R₂ is

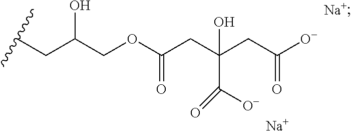

and positional isomers thereof.

In another embodiment, R is C₈-C₁₆ alkyl. In another embodiment, R is C₁₀-C₁₆ In another embodiment, R is one of C₈ alkyl, C₉ alkyl, C₁₀ alkyl, C₁₁ alkyl, C₁₂ alkyl, C₁₃ alkyl C₁₄ alkyl, C₁₅ alkyl, or C₁₆ alkyl.

Another embodiment is a personal care product comprising a surfactant composition of the present invention. In other aspects, the product is a shampoo, body wash, facial wash, or conditioning rinse.

Another embodiment of the present invention is a personal care product with reduced eye and skin irritation.

Another embodiment of the present invention is a surfactant composition wherein no salt for viscosity build is required.

Another embodiment of the present invention is a surfactant composition for use as a chelating agent. As one of ordinary skill in the art would appreciate, citric acid is a known, effective chelating agent.

Another embodiment of the present invention is a cleansing product, comprising a surfactant composition of the present invention. In aspects of the invention the cleansing product can be for use in hand wash and dish wash applications.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE INVENTION

Alkyl polyglucosides are complex products made by the reaction of glucose and fatty alcohol. In dealing with the chemistry one talks about degree of polymerization (the so called “d.p.”). In the case of traditional alkyl polyglucosides the d.p. is around 1.4. This means that on average there are 1.4 units of glucose for each alkyl group. The fact of the matter is that the resulting material is a mixture having an average of 1.4.

As one of ordinary skill in the art would understand, the specific structure of the an APG is sometimes difficult to ascertain completely since many positional isomers are possible, but two examples of structures are as follows:

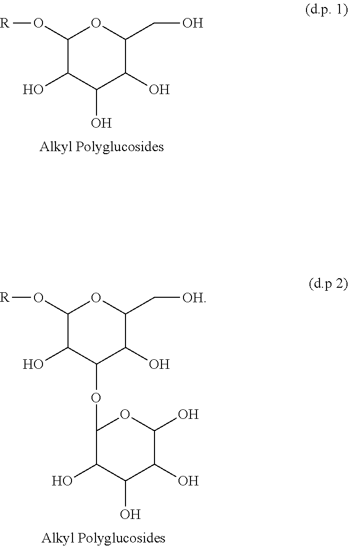

It should be clear that if there is a 50/50 mixture of the d.p. 1 and d.p. 2 product, the resulting analytical data will show that on average there is a d.p. of 1.5. Saying that a molecule has a d.p. of 1.5 does not mean that each molecule has 1.5 glucose units on it.

One aspect of the present invention relates to the heretofore unappreciated fact that the rather hydrophobic alkyl polyglycosides can contain on average five hydroxyl groups, one primary and the other four secondary. The assumption that there is a large degree of group specificity for the primary to react exclusively rather than the four additional hydroxyl groups is simply not true. This means that if on average only one of the five groups is reacted, there remains a very large concentration of reacting alkyl polyglycoside that has no functionality on it. Since the reactant with no functionalization remains rather water insoluble, there needs to be at least 2 and as many as 4 hydroxyl groups functionalized to get to the desired water-soluble product. We have observed that when between 2 and 5 groups are reacted, a water-soluble very useful product results. Therefore it is a preferred embodiment having between 2 and 5 of the hydroxyl groups functionalized.

Another aspect is that in making the compounds of the present invention is the selection of the proper reagents to make the desired product. Specifically, the reaction of the alkyl polyglycoside with a certain family of epoxy compounds and related materials occurs under mild aqueous conditions.

Examples of the compositions of the present invention are prepared by reacting the mixtures of polyglucosides, including those of the following structures and positional isomers thereof:

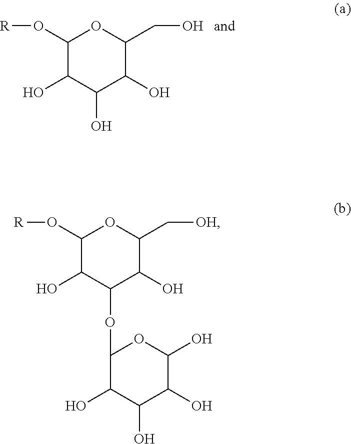

and positional isomers thereof, wherein R is alkyl having 1 to 24 carbon atoms;
with a functionalizing agent of the following formula:

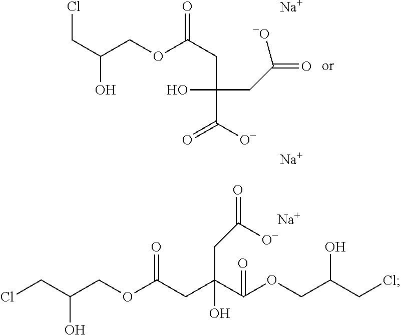

and optionally with a polymerizing agent, including one of the following structures:

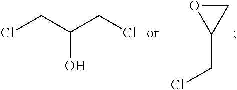 [SMILES: OC(CCl)CCl ; ClCC1CO1]

and NaOH and water.

As stated above, the compounds and compositions of the present invention are understood to comprise positional isomers thereof. Positional isomers of the present invention specifically include various attachment points of the epichlorohydrin to the citrate ion. Various examples of the positional isomers of the present invention are disclosed herein.

Examples of how these functioning agents can be made is shown in the schemes shown below. To make a di-epichlorohydrin adduct, it is possible to start with the mono-citrate anion (and thus, two acid groups). For the mono-epichlorohydrin adduct, it is possible to start with the di-citrate anion.

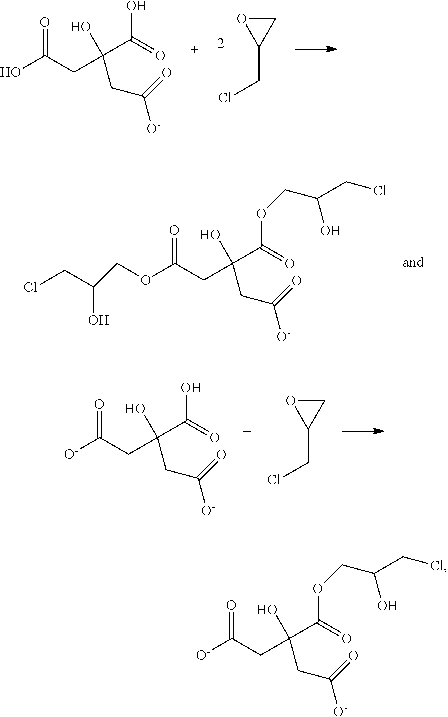 [SMILES: O=C([O-])CC(O)(CC(=O)O)C(=O)O ; ClCC1CO1 ; O=C([O-])CC(O)(CC(=O)OCC(O)CCl)C(=O)OCC(O)CCl ; O=C([O-])CC(O)(CC(=O)[O-])C(=O)O ; ClCC1CO1 ; O=C([O-])CC(O)(CC(=O)[O-])C(=O)OCC(O)CCl]

As used herein, the term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein.

Examples of alkyl groups of the present invention include C₁-C₂₄ alkyl, C₈-C₁₄ alkyl, C₈ alkyl, C₉ alkyl, C₁₀ alkyl, C₁₁ alkyl, C₁₂ alkyl, C₁₂ alkyl, and C₁₄ alkyl.

In addition to the citrate-functionalized alkyl polyglucosides described above, as mentioned previously, one aspect of the present invention relates to a functionalized polyglycoside derivative that is made by the polymerized reaction of a polyglycoside, together with a functionalizing agent that contains a citrate ester group. The preferred polymers are cross-linked having more than one group per molecule. When polymerizing alkyl polyglucosides with epichlorohydrin, there is not a great deal of group specificity in the reaction of the various hydroxyl groups and cross-linked polymers may result. The degree of cross linking depends upon the ratio of epichlorohydrin to hydroxyl groups chosen. The functionalizing agent likewise reacts with hydroxyl groups, providing a multifunctional polymer.

By considering the reaction in steps, it will make the reaction pathway clearer.

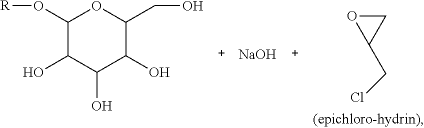

wherein R is alkyl.

Epichlorohydrin reacts with the first hydroxyl giving an intermediate:

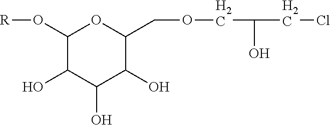

The above reaction shows one of the possible reacted hydroxyl groups. There is a potential for reaction of the other hydroxyl groups as well. Subsequently, another hydroxyl group reacts to give:

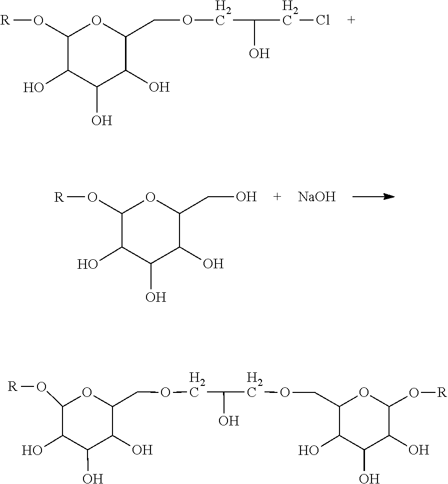

The functionalization group is added to one of the additional hydroxyl groups, for example:

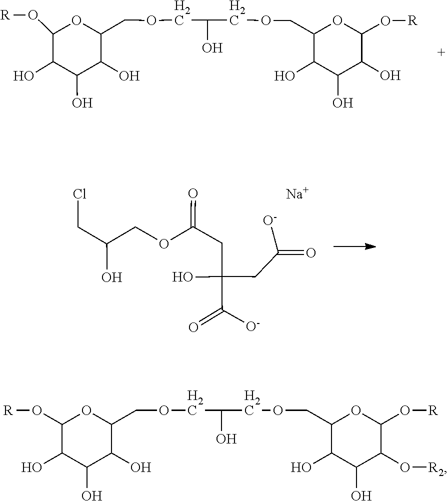

wherein R is alkyl and R₂ is:

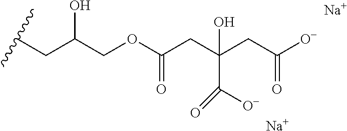 [SMILES: *CC(O)COC(=O)CC(O)(CC(=O)[O-])C(=O)[O-] ; [Na+] ; [Na+]]

As the reaction continues more and more hydroxyl groups react with either the polymerizing agent or the functionalizing agent. The structure is complicated not only by the fact that many hydroxyl groups can react with the different types of agent, but also by the fact that commercial polyglycosides are mixtures having an average dp of 1.5. The resulting products are hydroxypropyl cross linked polymers having branching present.

### Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

### Examples of Compounds of the Present Invention

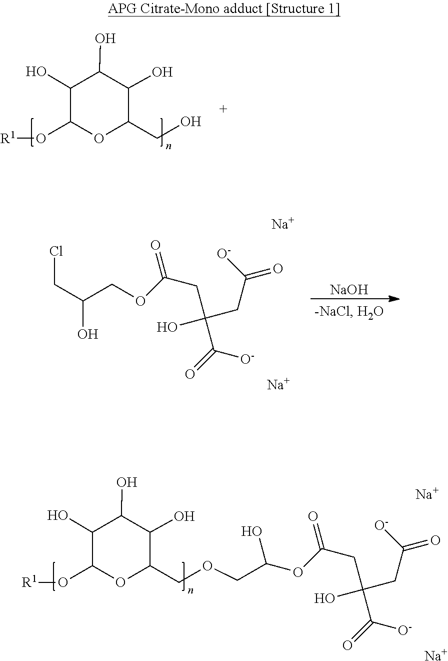

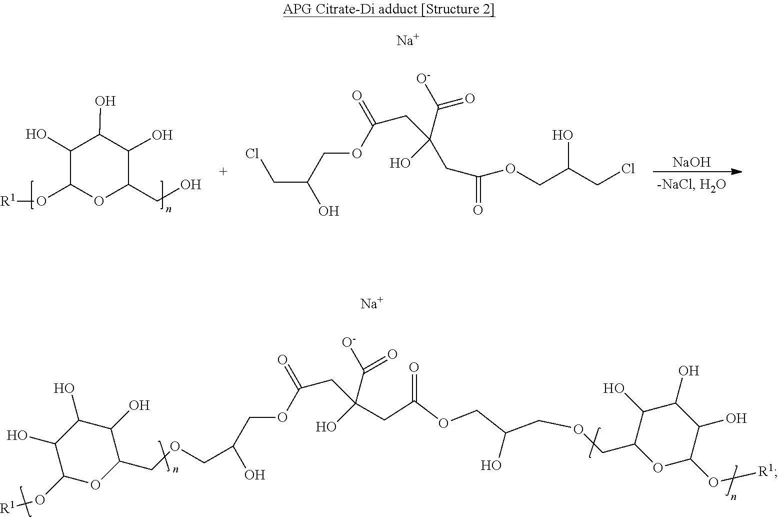

wherein R¹ is alkyl and n is an integer from 1 to 4; and positional isomers thereof.

Thus, one embodiment of the present invention is a composition comprising a component of the following formula:

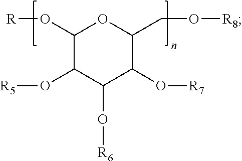

wherein R is a C₁-C₂₄ alkyl; n is an integer from 1 to 4; R₅, R₆, R₇, and R₈ are independently chosen from H or the following:

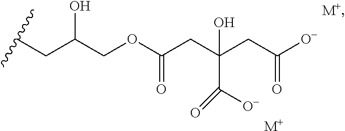

with the proviso that R₅, R₆, R₇, and R₈ are not all H; and M is a counterion such as Na, K, and NH₄; and positional isomers thereof.

Another embodiment of the present invention is a composition comprising a component of the following formula:

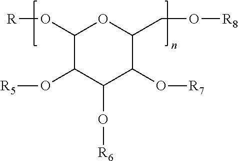

wherein R is a C₁-C₂₄ alkyl; n is an integer from 1 to 4; R₅, R₆, R₇, and R₈ are independently selected from H or the following:

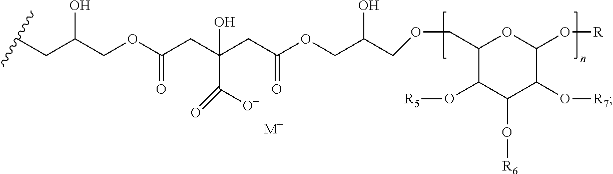

with the proviso that R₅, R₆, R₇, and R₈ are not all H; and M is selected from Na, K, and NH₄; and positional isomers thereof.

Another embodiment of the present invention is a surfactant composition of the following formula:

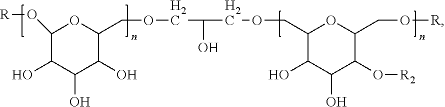

wherein R is C₁-C₂₄ alkyl and R² is

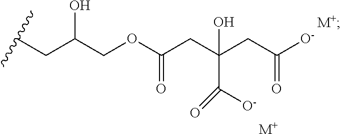

and n is an integer from 1 to 4; M is a counterion such as Na, K, and NH₄; and positional isomers thereof.

Another embodiment of the present invention is a surfactant composition of the following formula:

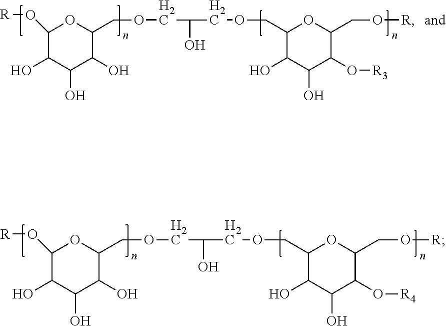

wherein R is C₁-C₂₄ alkyl; and R₃ and R₄ are attachment points for the following functionalizing linker group:

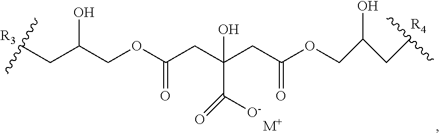

and M is a counterion such as Na, K, and NH₄; n is an integer from 1 to 4; and positional isomers thereof.

### Preparation of Epi-Citrate Functionalizing Agent (3-Chloro-2-Hydroxypropyl Citrate):

The reaction of epichlorohydrin with citric acid has been reported in the literature (C. He et al, Journal of Colloid and Interface Science, 454 (2015) 216-225; Japanese patent JP2015196675A; Polish patent, PL 136719). For the purposes of this invention, epichlorohydrin and citric acid were reacted according to the following procedure:

Water and citric acid are charged in a 250-mL round bottom flask. Sodium hydroxide (50%, 2 moles for every mole of citric acid) is charged and the mixture exotherms. After the mixture returns to room temperature, epichlorohydrin is added and the reaction is gradually brought to a temperature of 45° C. and maintained at that temperature for 6-8 hours until the mixture goes from hazy to clear. The reaction is complete when the pH and acid value are both stable.

Preparation of a APG Citrate Surfactant (Mono Adduct) of this Invention is as Follows (Disodium Laurylglucosides Hydroxypropyl Citrate)(Structure 1):

One equivalent of lauryl glucoside is charged to a reactor along with water. The mixture is heated to approximately 50° C. and then one equivalent of 3-chloro-2-hydroxypropyl citrate is charged slowly. One equivalent of NaOH (50%) is then charged over a period of about 2 hours, keeping the pH of the reaction between 8-10. The reactor temperature is then increased to 75-85° C. The progress of the reaction is monitored by the increase in ionic chloride ion. When the calculated level of chloride is reached, the reaction is cooled and the pH is adjusted to 6-8 with citric acid. Total Solids are adjusted by adding water.

### Preparation of Di-Epi-Citrate Functionalizing Agent (Also Mentioned in Japanese Patent JP2015196675A):

Water and citric acid are charged in a 250-mL round bottom flask. Sodium hydroxide (50%, one mole for every mole of citric acid) is charged and the mixture exotherms. After the mixture returns to room temperature, epichlorohydrin is added and the reaction is gradually brought to a temperature of 45° C. and maintained at that temperature for 6-8 hours until the mixture goes from hazy to clear. The reaction is complete when the pH and acid value are both stable.

Preparation of Di-APG-Citrate Surfactant (Diadduct) of this Invention is as Follows (Structure 2):

One equivalent of lauryl glucoside is charged to a reactor along with water. The mixture is heated to approximately 30-40° C. and then one molar chloride equivalent of Di-Epi-Citrate is charged. One equivalent of NaOH (50%) is then charged over a period of about 2 hours, keeping the pH of the reaction between 8-10. The reaction temperature is then increased to 75-85° C. The progress of the reaction is monitored by the increase in ionic chloride ion. When the calculated level of chloride is reached, the reaction is cooled and the pH is adjusted to 6-8 with citric acid. Total Solids are adjusted by adding water.

The above procedures describe the method for making citrate functionalized APG surfactants. The synthesis of these surfactants starts with alkyl polyglucosides (APG's). Other surfactants of the current invention can start with APG's that have first been cross-linked, with either epichlorohydrin or 1,3-dichloro-2-propanol, by methods described in U.S. Pat. No. 7,507,399 cited above. C12-PolyAPG referenced below is one such cross-linked APG that is made by crosslinking a C12-APG (Lauryl Glucoside) with itself using epichlorohydrin. This C12-PolyAPG is then reacted with the Epi-Citrate functionalizing groups of the current invention as described below.

### C12-PolyAPG+Mono-Epi-Citrate:

One equivalent of C12-PolyAPG is charged to a reactor along with water. The mixture is heated to approximately 50° C. and then one molar chloride equivalent of Mono-Epi-Citrate is charged. One equivalent of NaOH (50%) is then charged over a period of about 2 hours, keeping the pH of the reaction between 8-10. The reaction temperature is then increased to 75-85° C. The progress of the reaction is monitored by the increase in ionic chloride ion. When the calculated level of chloride is reached, the reaction is cooled and the pH is adjusted to 6-8 with citric acid. Total Solids are adjusted by adding water.

### C12-PolyAPG+Di-Epi-Citrate:

One equivalent of C12-PolyAPG is charged to a reactor along with water. The mixture is heated to approximately 50° C. and then one molar chloride equivalent of Di-Epi-Citrate is charged. One equivalent of NaOH (50%) is then charged over a period of about 2 hours, keeping the pH of the reaction between 8-10. The reaction temperature is then increased to 75-85° C. The progress of the reaction is monitored by the increase in ionic chloride ion. When the calculated level of chloride is reached, the reaction is cooled and the pH is adjusted to 6-8 with citric acid. Total Solids are adjusted by adding water.

The “citrate” functionalized APG's of the current invention perform as primary surfactants in shampoos, body washes and facial cleansers and other cleansing beauty products. They are 100% naturally derived, PEG-free and Dioxane-free, non-irritating to skin and eyes and ultimately biodegradable. The following performance data is meant to illustrate their properties in cleansing formulations.

### Performance Data

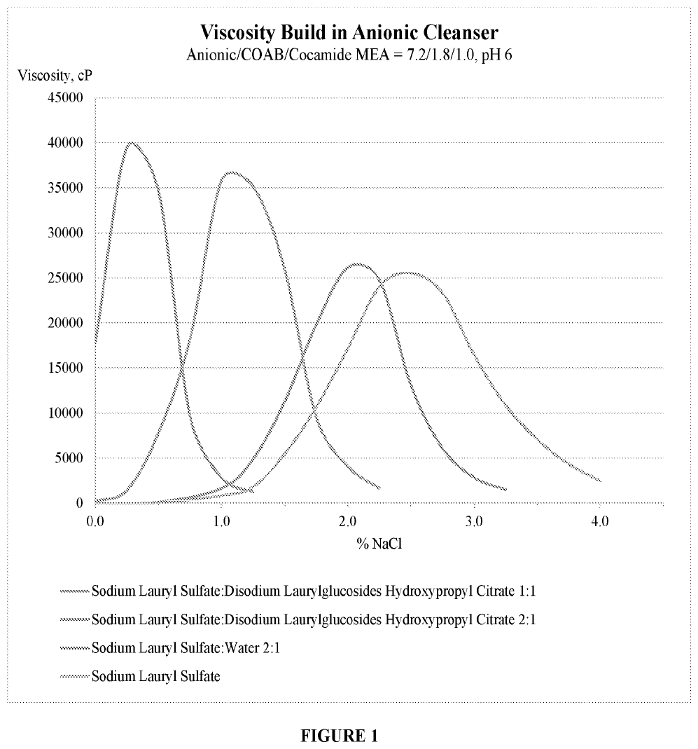

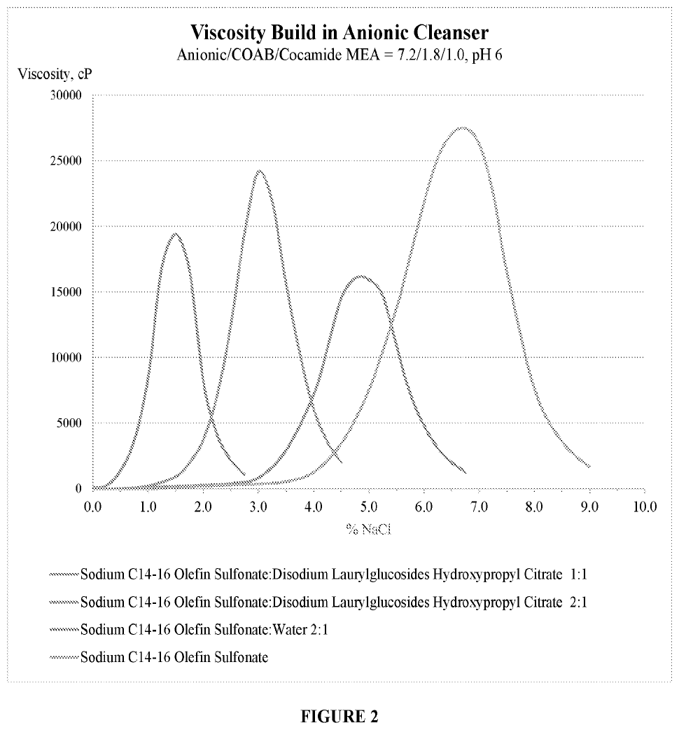

Anionic surfactants of the present invention were added to various cleansing formulations to demonstrate their benefits. The generalized cleansing formulations are shown in Table 1, where various anionic surfactants, or blends of anionic surfactants, were added to a betaine and an alkanolamide surfactant and water. The active levels of the various surfactants are given in Table 1, below. The balance of the weight in these formulas is water. Salt curves for these cleansing formulations were generated and are shown in FIGS. 1 and 2. These salt curves demonstrate the ability of the surfactants of this invention to support viscosity build in cleansers. Salts curves were generated for both sulfate-free blends based on AOS, Sodium C14-16 Olefin Sulfonate (FIG. 2), as well as sodium lauryl sulfate-based blends (FIG. 1). The salt curves in FIG. 1 are for the following: 1) Sodium Lauryl Sulfate (SLS) alone: 2) Sodium Lauryl Sulfate:Water, 2:1; 3) Sodium Lauryl Sulfate:Disodium Laurylglucosides Hydroxypropyl Citrate 2:1; and 4) Sodium Lauryl Sulfate:Disodium Laurylglucosides Hydroxypropyl Citrate, 1:1. The salt curves in FIG. 2 are for the following: 1) Sodium C14-16 Olefin Sulfonate (AOS) alone: 2) Sodium C14-16 Olefin Sulfonate:Water, 2:1; 3) Sodium C14-16 Olefin Sulfonate:Disodium Laurylglucosides Hydroxypropyl Citrate, 2:1; and 4) Sodium C14-16 Olefin Sulfonate:Disodium Laurylglucosides Hydroxypropyl Citrate, 1:1.

TABLE 1
Formulations to demonstrate the thickening of
anionic surfactants of the present invention

| Ingredient | Active Level |
|---|---|
| Anionic surfactant (SLS or AOS, alone or | 7.2 |
| blended with invention surfactants) | |
| Cocamidopropyl Betaine | 1.8 |
| Cocamide MEA | 1.0 |

Inspection of FIGS. 1 and 2 clearly show that surfactants of the current invention, for example, Disodium Laurylglucosides Hydroxypropyl Citrate, when incorporated into typical cleansing formulations, result in fast and high viscosity build. Some formulations can achieve desired viscosity levels without any added salt, meeting the demand for that type of claim by some shampoo manufacturers.

Formulation 1 below is an example of a fully formulated shampoo containing Disodium Laurylglucosides Hydroxypropyl Citrate of the present invention. It is a good example of how surfactants of the present invention can act as mild, primary surfactants in personal care cleansing products.

FORMULATION 1. Clean and Simple Shampoo

| Order | INCI Name | Trade Name | % | Function |
|---|---|---|---|---|
| 1 | Water | Water | qs | Carrier |
| | | | to 100.00 | |
| 2 | Polyquaternium- | UCARE ™ | 0.20 | Conditioning |
| | 10 | Polymer | | |
| | | JR-30M ¹ | | |
| 3 | Cocamidopropyl | Cola ®Teric | 7.00 | Viscosity |
| | Hydroxysultaine | CBS | | build |
| 4 | Disodium | Suga ®Citrate | 13.00 | Ultra-mild |
| | Laurylglucosides | L1C | | primary |
| | Hydroxypropyl | | | surfactant |
| | Citrate | | | |
| 5 | Disodium Lauryl | Cola ®Mate | 12.00 | Foam boosting |
| | Sulfosuccinate | LA-40 | | co-surfactant |
| 6 | Polyquaternium- | Poly | 2.00 | Conditioning, |
| | 81 | Suga ®Quat | | increases |
| | | S1210P | | deposition |
| | | | | of PQ-10 |
| 7 | Fragrance | Fragrance ² | 0.30 | Fragrance |
| 8 | Benzyl Alcohol | Euxyl ® K | 0.70 | Preservative |
| | and Ethylhexyl- | 900 ³ | | |
| | glycerin and | | | |
| | Tocopherol | | | |

### Typical Properties

| Appearance | Clear, Viscous Liquid |
|---|---|
| pH | 5.0-5.5 |
| Viscosity | 5,000-10,000 cP |
| Surfactant Solids | 13.6-14.5% |

### Zein Irritation Testing, Reduction of Surfactant Irritation by Disodium Laurylglucoside Hydroxypropyl Citrate

Surfactants of the current invention were tested for their ability to reduce the irritation potential of cleansing formulations. We used the Zein Test [referenced in J. Surfact. Deterg., A. Seweryn et al., (2019), 22: 743-750] to demonstrate this property of the surfactants.

The Zein test is intended for determining the irritation potential of a surfactant or surfactant-based product. Zein, a corn protein similar to keratin present in the skin and hair, is insoluble in water unless it is denatured by an irritant. In this test, the irritant is a surfactant or surfactant-based product, and irritation potential is predicted by the amount of zein solubilized.

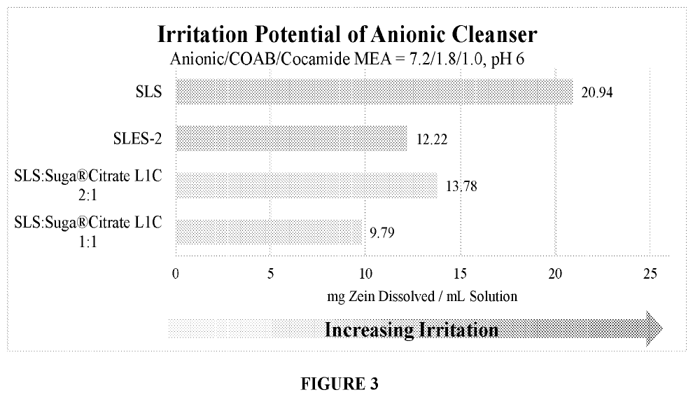

The surfactants of the current invention, for example Disodium Laurylglucosides Hydroxypropyl Citrate, are effective at reducing irritation potential in cleansing formulations, without negatively impacting other important attributes. The study shown in FIG. 3 demonstrates that the milder but undesirable Sodium Laureth-2 Sulfate (SLES) can be replaced with a combination of Sodium Lauryl Sulfate and disodium laurylglucosides hydroxypropyl citrate (Suga®Citrate LIC, sold by Colonial Chemical, Inc.). This combination exhibits lower irritation with even better viscosity building performance.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers. As stated above, the compounds described herein specifically include positional isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

The invention thus being described, it will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only.

1. A composition comprising a component of the following formula: 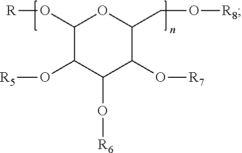
wherein R is a C₁-C₂₄ alkyl;
n is an integer from 1 to 4;
R₅, R₆, R₇, and R₈ are independently chosen from H or the following: 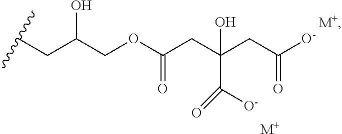
with the proviso that R₅, R₆, R₇, and R₈ are not all H; and
M is selected from Na, K, and NH₄; and positional isomers thereof.

2. A composition comprising a component of the following formula: 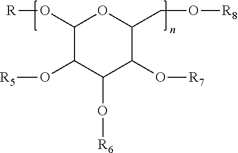
wherein R is a C₁-C₂₄ alkyl;
n is an integer from 1 to 4;
R₅, R₆, R₇, and R₈ are independently selected from H or the following: 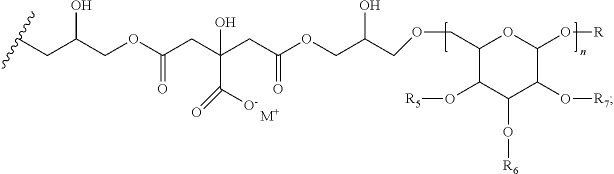
with the proviso that R₅, R₆, R₇, and R₈ are not all H; and
M is selected from Na, K, and NH₄; and positional isomers thereof.

3. A surfactant composition of the following formula: 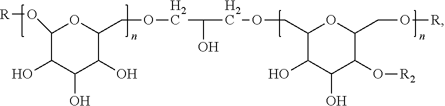
wherein R is C₁-C₂₄ alkyl and R² is 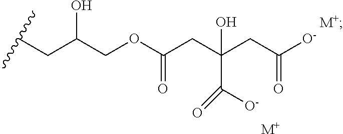
and
n is an integer from 1 to 4; M is selected from Na, K, and NH₄; and positional isomers thereof.

4. A surfactant composition of the following formula: 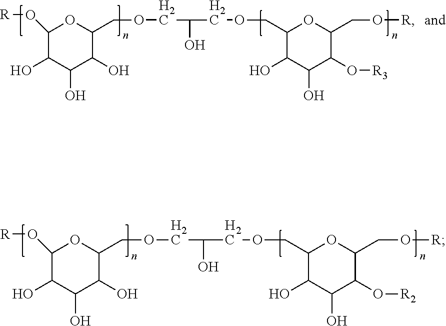
wherein R is C₁-C₂₄ alkyl; and R₃ and R₄ are attachment points for the following functionalizing linker group: 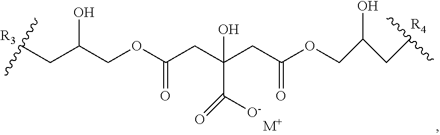
and M is selected from Na, K, and NH₄; n is an integer from 1 to 4; and positional isomers thereof.

5. A composition of claim 1, wherein R is C₈-C₁₆ alkyl.

6. A composition of claim 2, wherein R is C₈-C₁₆ alkyl.

7. A composition of claim 3, wherein R is C₈-C₁₆ alkyl.

8. A composition of claim 4, wherein R is C₈-C₁₆ alkyl.

9. A composition of claim 3, wherein R is C₁₀-C₁₆ alkyl.

10. A composition claim 4, wherein R is chosen from C₁₀ alkyl, C₁₁ alkyl, C₁₂ alkyl, C₁₃ alkyl, C₁₄ alkyl, C₁₅ alkyl, or C₁₆ alkyl.

11. A composition prepared by reacting the following:
(i) at least one compound of the following formula: 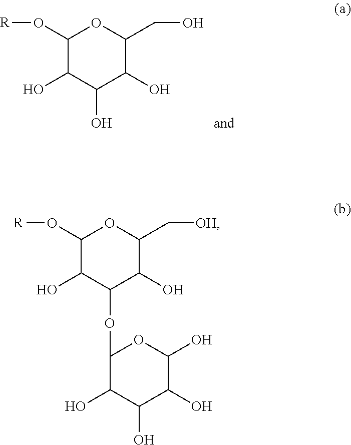
wherein R is an alkyl having 1 to 24 carbons; and
(ii) at least one functionalizing agent of the following formula 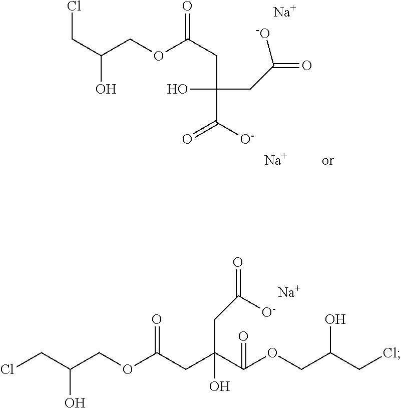
in water with NaOH at a temperature of between 75-85° C., for between 5-8 hours.

12. The composition of claim 11, prepared by further reacting with at least one polymerizing agent of the following formula: 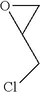
or Cl—CH₂CH(OH)—CH₂—Cl.

13. A personal care product, comprising a composition of claim 1.

14. A personal care product, comprising a composition of claim 2.

15. A personal care product, comprising a composition of claim 3.

16. A personal care product, comprising a composition of claim 4.

17. (canceled)

18. The personal care product of claim 1, in the form of a shampoo, body wash, facial wash, or conditioning rinse.

19. (canceled)

20. The personal care product of claim 13, wherein the level of eye and skin irritation is reduced.

21. The personal care product of claim 13, wherein no salt for viscosity build is required.

22. A composition of claim 1, for use as a chelating agent.

23. A cleansing product, comprising a composition of claim 1, for use in hand wash and dish wash applications.
